(19) United States
(12) Patent Application Publication
(10) Pub. No.: US 2025/0135179 A1
(43) Pub. Date: May 1, 2025

(54) CONNECTION TOOL FOR A CONNECTING DEVICE

(71) Applicant: MSD International GmbH, Luzern (CH)

(72) Inventors: Brian MULLARKEY, Carlow (IE); Noel LAHART, Clonmel, Co Tipperary (IE); John HAYES, Clonmel, Co Tipperary (IE)

(73) Assignee: MSD International GmbH, Luzern (CH)

(21) Appl. No.: 18/877,697

(22) PCT Filed: Jul. 18, 2023
(86) PCT No.: PCT/IB2023/000426

§ 371 Date: Dec. 20, 2024

Related U.S. Application Data
(60) Provisional application No. 63/391,461, filed on Jul. 22, 2022.

(51) Int. Cl. A61M 39/18; A61M 39/16

(52) U.S. Cl. CPC A61M 39/18; A61M 39/165

(57) ABSTRACT

A connection tool for use in connecting tubing lines is provided. The connection tool includes a housing having a first and second housing portions. The first housing portion includes a first central recess for receiving a body therein, and a first slide track portion extending from the first central recess. The second housing portion is movable relative to the first housing portion and includes a second central recess for receiving the body therein, and a second slide track portion extending from the second central recess. The first and second slide track portions form a sliding handle track when the first and second housing portions are moved to a closed position.

### BACKGROUND OF THE DISCLOSURE

Connecting devices are used to connect e.g., tubing lines together. Such tubing lines can be sterile or non-sterile fluid lines. Certain connecting devices have protective sheets that cover openings to preserve sterility or to maintain cleanliness that must be removed prior to use. In doing so, typical protective sheets are pulled off via a pull tab mechanism. However, and especially in dealing with sterile fluid lines, it is critical that the protective sheets be pulled off properly. This is typically done by pulling the pull tab precisely in a linear manner in a direction perpendicular to a direction of flow of the fluid lines being connected and without movement of the connecting device during the pulling movement to remove the pull tab. The protective sheets must also be pulled off without any horizontal or vertical movement, or play of the fluid lines that is connected to the connecting device. If the protective sheets are not precisely pulled off in the proper manner, leaks and/or loss of sterility results within the connecting device. As such, there is a need to address the problems associated with connecting devices for fluid lines having protective sheets that must be removed.

### BRIEF SUMMARY OF THE DISCLOSURE

In accordance with an exemplary embodiment of the subject disclosure there is provided a connection tool comprising a housing that comprises: a first housing portion having: a first central recess for receiving a body therein, and a first slide track portion extending from the first central recess, a second housing portion movable relative to the first housing portion, the second housing portion having: a second central recess for receiving the body therein, and a second slide track portion extending from the second central recess, wherein the first and second slide track portions form a sliding handle track upon the first and second housing portions being moved to a closed position.

In certain exemplary embodiments, the first housing portion further comprises: a first halfpipe in fluid communication with the first central recess; and a second halfpipe in fluid communication with the first central recess. The second housing portion further comprises: a first halfpipe in fluid communication with the second central recess; and a second halfpipe in fluid communication with the second central recess, wherein the first halfpipes of the first and second housing portions form an annular pipe when the first and second housing portions are moved to a closed position, and the second halfpipes of the first and second housing portions form an annular pipe when the first and second housing portions are moved to the closed position.

In certain exemplary embodiments, the first slide track portion has a longitudinal axis that extends substantially perpendicular to a longitudinal axis of one of the first or second halfpipes of the first housing portion. The first slide track portion comprises a slot extending along its entire longitudinal extent. The first and second housing portions are pivotably connected.

In certain exemplary embodiments, the housing further comprising a hinge operatively connected to the first housing portion and the second housing portion. The housing further comprising a living hinge operatively connected to the first housing portion and the second housing portion. The first housing portion comprises a detent and the second housing portion comprises a cooperating detent for releasably engaging the detent of the first housing portion. The first housing portion comprises a wing adjacent the first slide track portion. The first housing portion comprises a left wing and a right wing, each adjacent the first slide track portion.

In certain exemplary embodiments, the connection tool further comprises a slide handle slidable within the sliding handle track. The connection tool further comprises a slide handle that comprises: a main body having a recess for receiving a device therein; a paw spaced from the recess of the main body; and a handle spaced from the main body.

In certain exemplary embodiments, the paw is a hook or tab. The slide handle further comprising a rail having a width larger than a width of the main body. The slide handle comprises: a main body having a recess, and wherein the main body is sized to pass through the slot of the first slide track portion; a paw spaced from the recess of the main body; and a handle spaced from the main body.

In accordance with another exemplary embodiment of the subject disclosure there is provided a connection tool comprising a housing that comprises: a first housing portion having: a first central recess for receiving a body therein, a first halfpipe in fluid communication with the first central recess, a second halfpipe in fluid communication with the first central recess, a first slide track portion extending substantially transverse to a central axis of the first central recess, and a wing adjacent the first slide track portion, a second housing portion pivotably connected to the first housing portion, the second housing portion having: a second central recess for receiving the body therein, a first halfpipe in fluid communication with the second central recess, a second halfpipe in fluid communication with the second central recess, a second slide track portion extending substantially transverse to a central axis of the second central recess, and a wing adjacent the second slide track portion, wherein the first and second slide track portions form a sliding handle track when the first and second housing portions are connected; and a slide handle that comprises: a main body having a recess for receiving a device therein, and wherein the main body is configured to pass along the sliding handle track, a paw spaced from the recess of the main body, a handle spaced from the main body, and a rail adjacent the main body.

In accordance with yet another exemplary embodiment of the subject disclosure there is provided a connection tool comprising a housing that comprises: a recess for receiving a body therein, an annular tube in fluid communication with the recess, and a sliding handle track extending substantially transverse to a longitudinal central axis of the annular tube; and a slide handle slidable along the sliding handle track.

In accordance with another exemplary embodiment of the subject disclosure there is provided a method of connecting tubes together comprising: connecting a first tube to a connection device having a pull tab; connecting a second tube to the connection device; attaching a connection tool to the connection device, the connection tool having a housing for housing the connection device, and a sliding handle track; attaching a slide handle to the pull tab; pulling the pull tab with the slide handle along the sliding handle track.

Other features and advantages of the subject disclosure will be apparent from the following more detailed description of the exemplary embodiments.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown in the drawings exemplary embodiments. It should be understood, however, that the subject application is not limited to the precise arrangements and instrumentalities shown.

FIG. 1 is perspective view of a connection tool in accordance with an exemplary embodiment of the subject disclosure;

FIG. 2 is a perspective view of a housing of the connection tool of FIG. 1;

FIG. 3 is a perspective view of a first housing portion of the connection tool of FIG. 1;

FIG. 4 is a top plan view of a first housing portion of the connection tool of FIG. 1;

FIG. 5 is a side elevational view of a first housing portion of the connection tool of FIG. 1;

FIG. 6 is a bottom plan view of a first housing portion of the connection tool of FIG. 1;

FIG. 7 is a perspective view of a second housing portion of the connection tool of FIG. 1;

FIG. 8 is a top plan view of a second housing portion of the connection tool of FIG. 1;

FIG. 9 is a side elevational view of a second housing portion of the connection tool of FIG. 1;

FIG. 10 is a bottom plan view of a second housing portion of the connection tool of FIG. 1;

FIG. 11 is a perspective view of a living hinge embodiment of the connection tool of FIG. 1;

FIG. 12 is a perspective view of a prior art connection device appliable to the connection tool of the subject disclosure;

FIG. 13 is a perspective view of the connection tool of FIG. 1 in an open position;

FIG. 14 is a perspective view of the connection tool of FIG. 1 in a closed position;

FIG. 15 is a perspective view of a slide handle of the connection tool of FIG. 1;

FIG. 16 is a side view of the slide handle of FIG. 15;

FIG. 17 is a perspective view of another exemplary embodiment of a slide handle applicable to the connection tool of FIG. 1;

FIG. 18 is a perspective view of a connection device seated in the connection tool of FIG. 1; and

FIG. 19 is a perspective view of a slide handle engaged with a pull tab of a connection device appliable to the present disclosure.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure, and that various features of the invention can be found throughout the various exemplary embodiments.

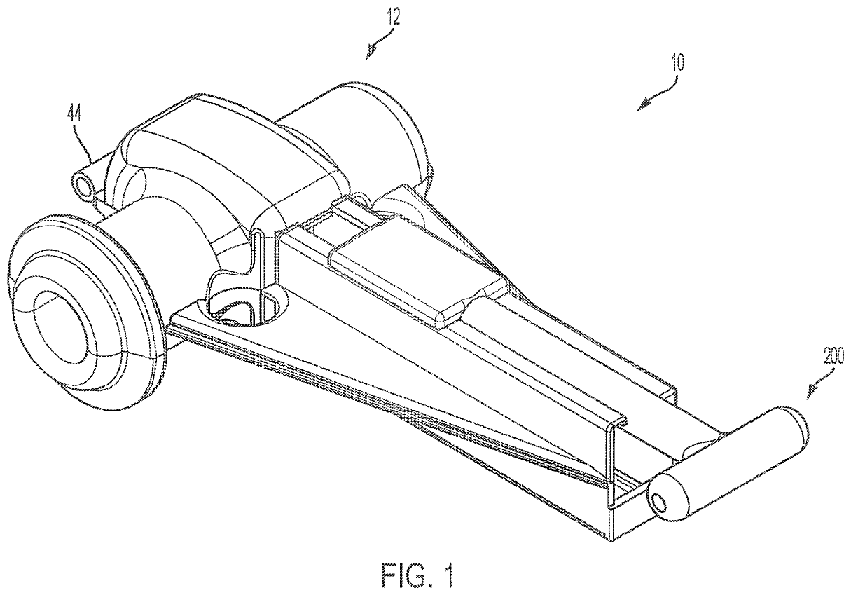

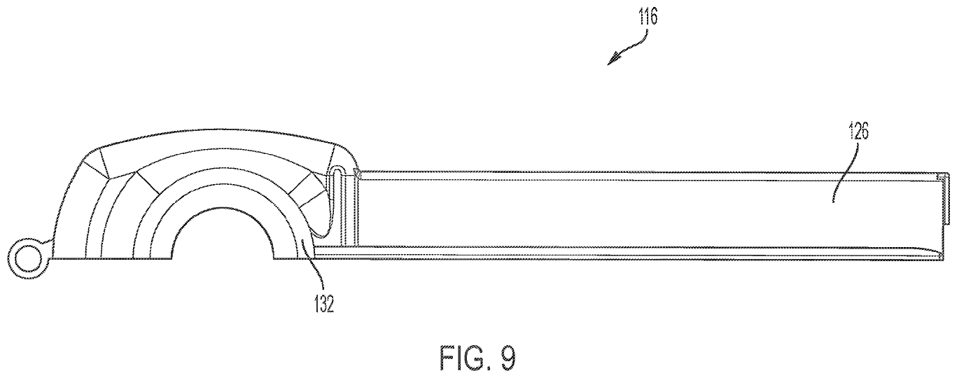

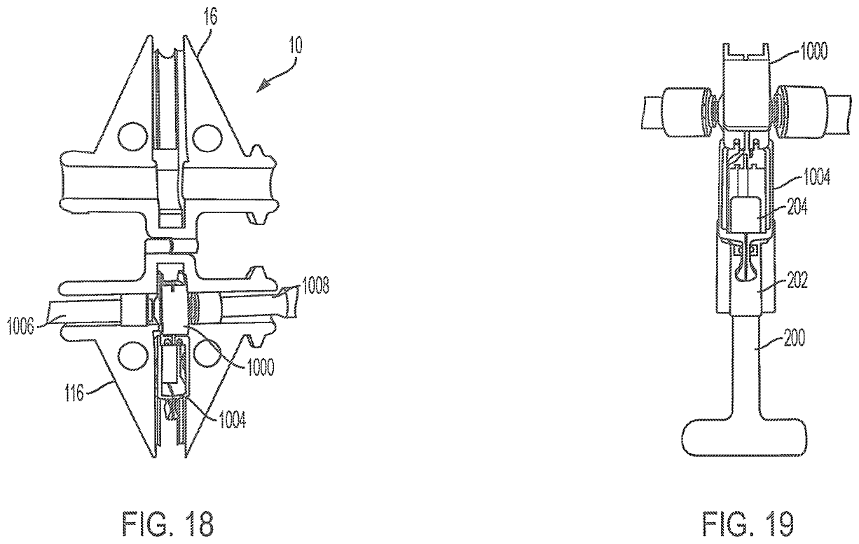

In accordance with an exemplary embodiment, the subject disclosure provides a connection tool 10 having a housing 12 and a slide handle 200, as shown in FIGS. 1-11 and 13-19. The connection tool is configured as shown in FIGS. 1-11 and 13-19. The slide handle 200 is configured to operatively engage the housing, as further discussed below.

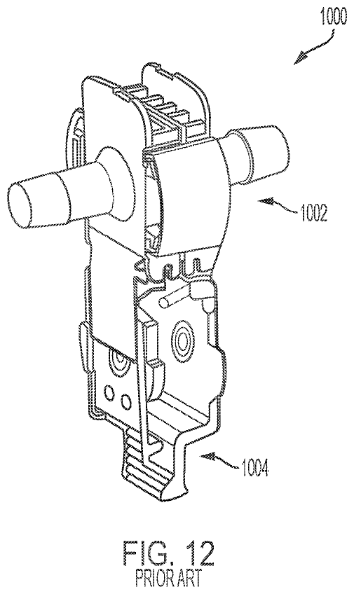

The housing 12 includes a first housing portion 16 and a second housing portion 116. The first housing portion 16 is configured as shown in FIGS. 1-6 and includes a first central recess 20 about a midportion of the first housing portion, and a first slide track portion 22. The first central recess is configured e.g., sized and shaped to receive a body therein. The body can be a body portion 1002 of a connecting device, such as the AseptiQuik® Sterile Connector 1000 by Colder Products Company 1000 (FIG. 12).

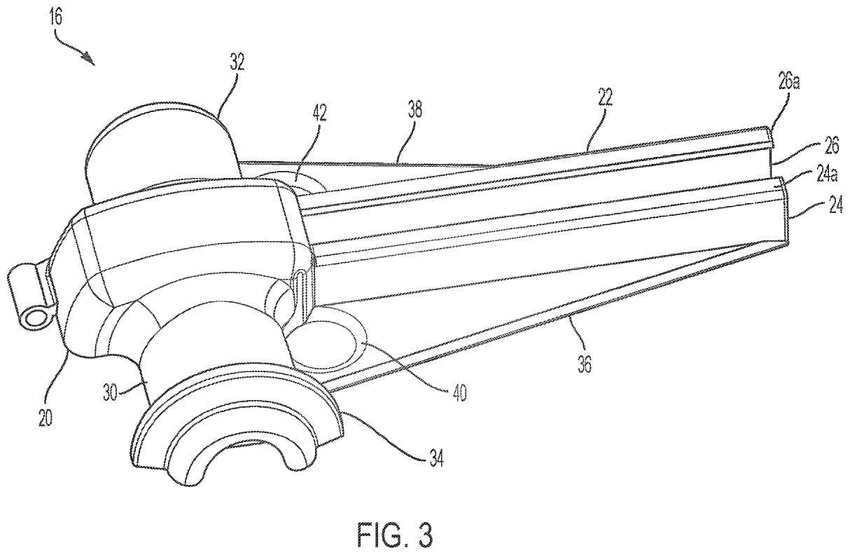

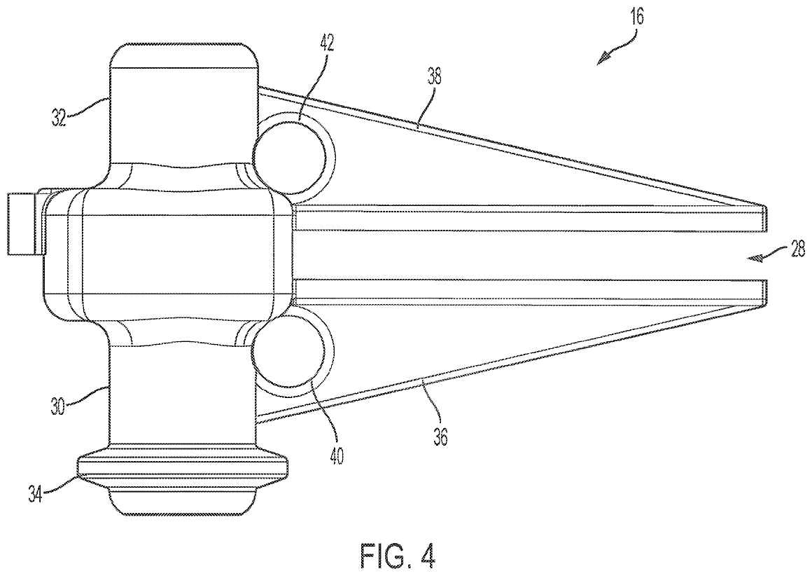

Referring to FIGS. 3 and 4, the first slide track portion 22 is an elongated track extending from the first central recess. The first slide track portion 22 includes a pair of lateral walls (i.e., a first lateral wall 24, and a second lateral wall 26) spaced apart, forming a slot 28 (FIG. 4) therebetween. The slot 28 extends along the entire length or longitudinal extent of the first slide track portion. Each of the lateral walls includes an inwardly or medially directed flange 24a, 24b, respectively. Each flange 24a, 24b extends an entirety of the longitudinal length or longitudinal extent of the first slide track portion.

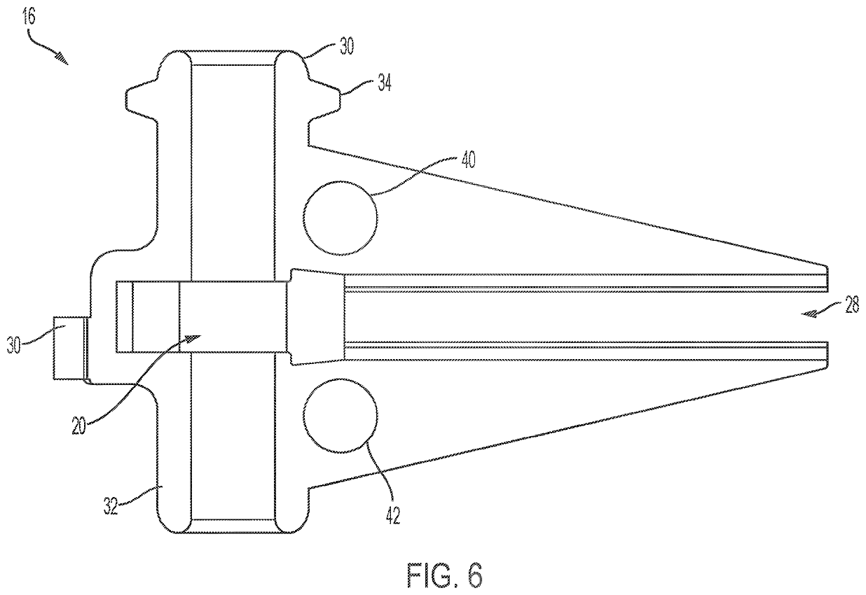

The first housing portion 16 further includes a first halfpipe 30 and a second halfpipe 32. The first and second halfpipes extend laterally from the first central recess 20 (FIG. 6) about opposite lateral sides of the first central recess. That is, each of the first and second halfpipes have a longitudinal axis or central longitudinal axis that extends substantially perpendicularly to a longitudinal extent of the first central recess 20 or a longitudinal axis of the first slide track portion. The central longitudinal axis of the first halfpipe 30 is also positioned to be coaxial with the central longitudinal axis of the second halfpipe 32. Each of the first and second halfpipes extends a length greater than a width of the first central recess 20 and less than an overall length of the first slide track portion 22. The first halfpipe 30 also includes a radially extending flange or rib 34 (FIG. 4) adjacent its lateral end.

Furthermore, the first housing portion 16 includes a first wing 36 (FIGS. 3 and 4) adjacent the first lateral wall 24 and a second wing 38 adjacent the second lateral wall. In other words, the first wing is adjacent the first slide track portion and the second wing is adjacent the first slide track portion. The first wing 36 is substantially triangular in shape and substantially planar. The first wing extends from the first slide track portion, specifically the first lateral wall 24, and the first halfpipe 30. Additionally, the first wing includes a through hole 40, sized and shaped to receive the passage of a human finger therethrough.

The second wing 38 is similarly configured as the first wing 36. The second wing 38 is substantially triangular in shape and substantially planar. The second wing extends from the first slide track portion, specifically the second lateral wall 26, and the second halfpipe 32. Additionally, the second wing includes a through hole 42, sized and shaped to receive the passage of a human finger therethrough.

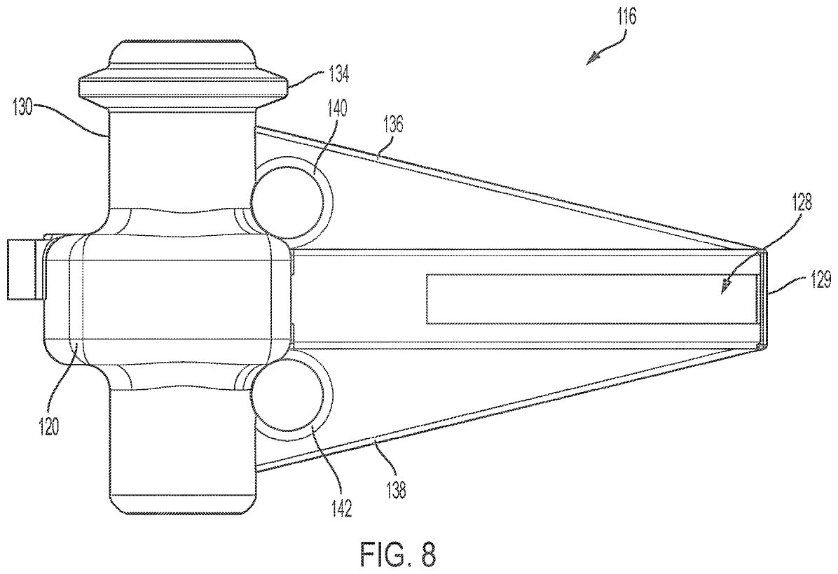

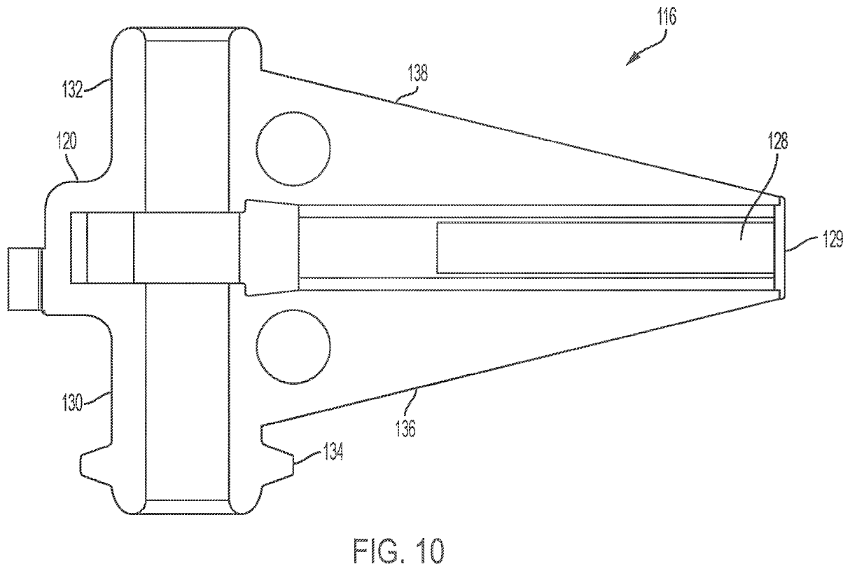

The second housing portion 116 is configured as shown in FIGS. 1, 2 and 7-10 and is movable relative to the first housing portion. The second housing portion includes a second central recess 120 (FIGS. 8 and 10) about a midportion of the second housing portion, and a second slide track portion 122. The second central recess is configured e.g., sized and shaped to receive a body therein. The body can be a body portion 1002 of a connecting device, such as the AseptiQuik® Sterile Connector 1000 by Colder Products Company (FIG. 12).

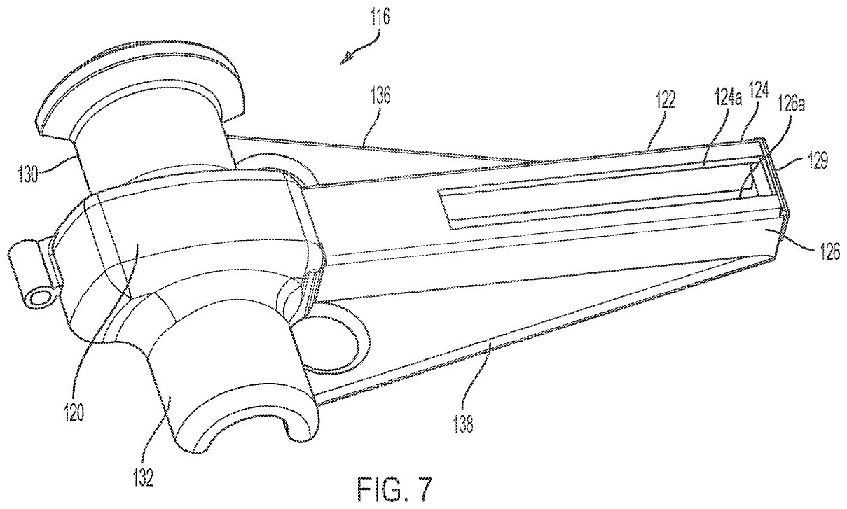

Referring to FIG. 7, the second slide track portion 122 is an elongated track extending from the second central recess. The second slide track portion 122 includes a pair of lateral walls (i.e., a first lateral wall 124, and a second lateral wall 126) spaced apart, forming a slot 128 (FIG. 8) therebetween. The slot 128 extends less than an entire length or longitudinal extent of the second slide track portion, e.g., about ½, ⅔, or ¾ of an overall length of the second slide track portion. Alternatively, the slot can be formed to extend an entire length or longitudinal extent of the second slide track portion. The second slide track portion also includes a stop 129 about its proximal end. The stop can be configured as a wall extending between the first and second lateral walls of the second slide track portion. Each of the lateral walls includes an inwardly or medially directed flange 124a, 124b, respectively. Each flange 124a, 124b extends an entirety of the longitudinal length or longitudinal extent of the slot 128.

Referring to FIGS. 8 and 10, the second housing portion 116 further includes a first halfpipe 130 and a second halfpipe 132. The first and second halfpipes extend laterally from the second central recess 120 about opposite lateral sides of the second central recess. That is, each of the first and second halfpipes have a longitudinal axis or central longitudinal axis that extends substantially perpendicularly to a longitudinal extent of the second central recess 120 or a longitudinal axis of the second slide track portion. The central longitudinal axis of the first halfpipe 130 is also positioned to be coaxial with the central longitudinal axis of the second halfpipe 132. Each of the first and second halfpipes extends a length greater than a width of the second central recess 120 and less than an overall length of the second slide track portion 22. The first halfpipe 130 also includes a radially extending flange or rib 134 adjacent its lateral end.

Furthermore, the second housing portion 116 includes a first wing 136 adjacent the first lateral wall 124 and a second wing 138 adjacent the second lateral wall 126 (FIG. 7). In other words, the first wing is adjacent the second slide track portion and the second wing is adjacent the second slide track portion. The first wing 136 is substantially triangular in shape and substantially planar, and substantially the same shape or profile as the first wing 36 of the first housing portion. The first wing extends from the second slide track portion, specifically the first lateral wall 124, and the first halfpipe 130. Additionally, the first wing includes a through hole 140, sized and shaped to receive the passage of a human finger therethrough.

Referring to FIG. 8, the second wing 138 is similarly configured as the first wing 136, and substantially the same shape or profile as the second wing 38 of the first housing portion. The second wing 138 is substantially triangular in shape and substantially planar. The second wing extends from the second slide track portion, specifically the second lateral wall 126, and the second halfpipe 132. Additionally, the second wing includes a through hole 142, sized and shaped to receive the passage of a human finger therethrough.

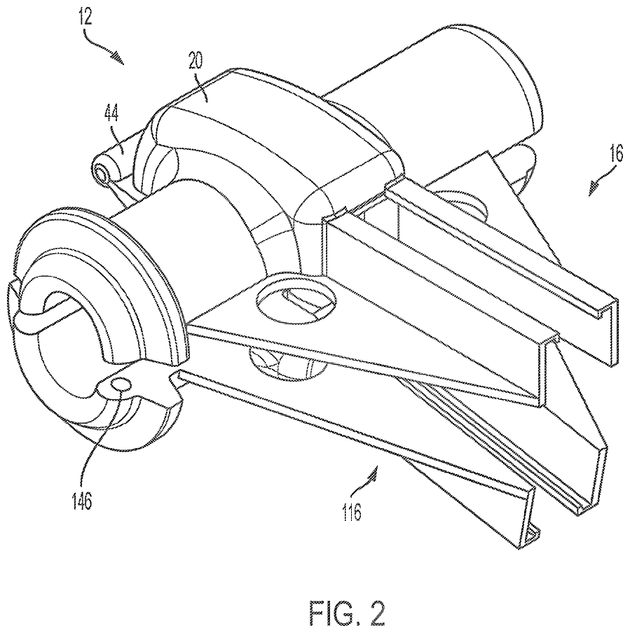

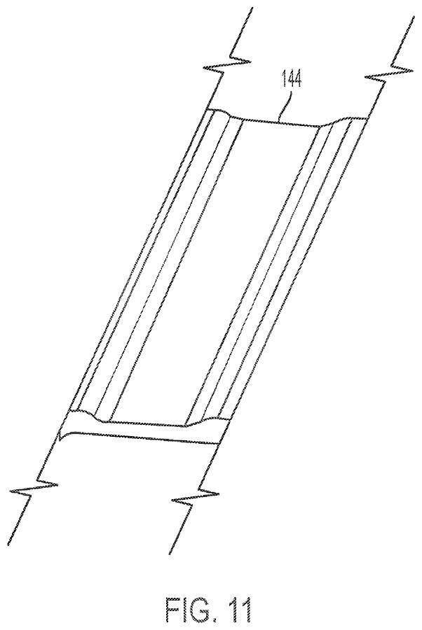

The housing 12 further includes a hinge 44 (FIG. 1) operatively connected to the first housing portion 16 and the second housing portion 116. The hinge 44 can be a living hinge 144 (FIG. 11) or any other hinge known in the art and applicable to the present disclosure. Owing to the hinge 44, the first housing portion 16 is pivotably connected to the second housing portion, as best shown in FIG. 2. The hinge is positioned about or adjacent the first and second central recesses 20, 120, defining a posterior most part of the housing.

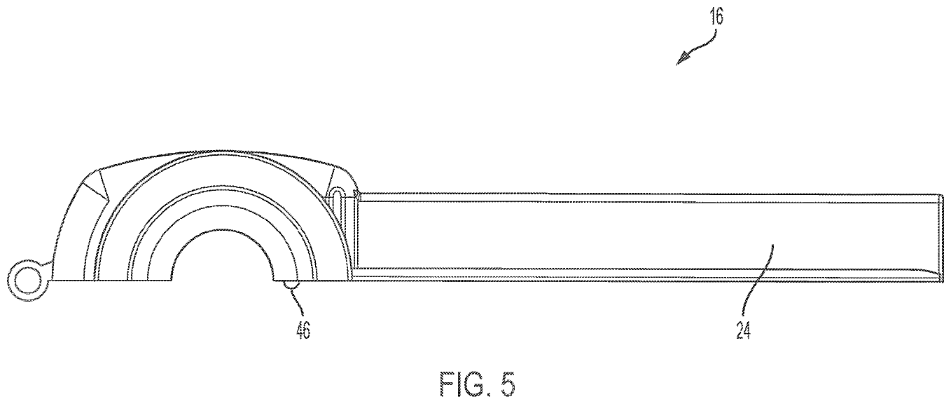

In accordance with another aspect of the exemplary embodiment, the first housing portion 16 can include a detent 46 (FIG. 5) configured to releasably engage a cooperating detent 146 (FIG. 2) on the second housing portion 116. The detent can be a snap-fit, ball and socket detent, or the like suitable for the intended use of the present exemplary embodiment.

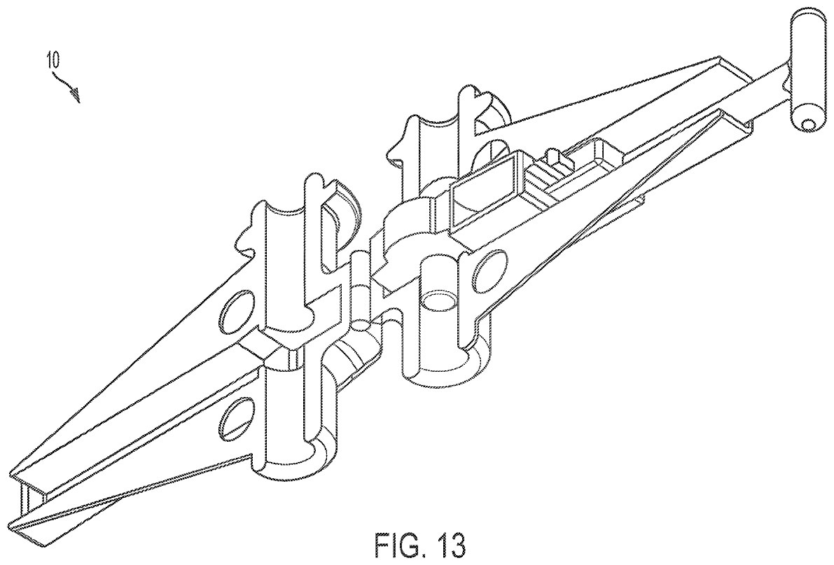

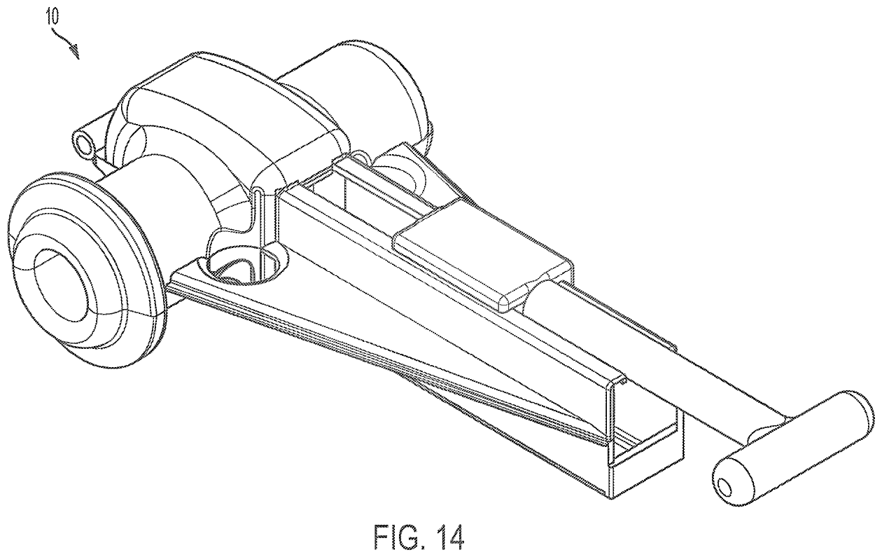

Owing to the hinge 44, the first and second housing portions are movable between a first position (FIG. 13) and a second position (FIG. 14). The first position is an open position to allow a user to attach the connection tool 10 to a connecting device or connection device, such as the AseptiQuik® Sterile Connector 1000 by Colder Products Company 1000 (FIG. 12). The connection tool is attached or secured to the connecting device 1000 by moving the first and second housing portions to the second position, thereby clamping the connection tool to the connecting device. By clamping the first and second housing portions of the connection tool to the connecting device, the connecting device is securely held in place with the connection tool. The first and second housing portions can be sized and/or configured such that clamping together provides a clamping force on the connecting device, or alternatively the first and second housing portions can be sized and/or configured such that clamping together exerts no clamping forces on the connecting device, or alternatively the first and second housing portions can be sized such that the first and second central recesses provides a clamping force on the connecting device while the first and second halfpipes exert no clamping forces on the connecting device or the tubing lines connected by the clamping device.

When the first and second housing portions are brought together or connected together (FIG. 14), the first halfpipes of the first and second housing portions form an annular pipe (e.g., a first annular pipe), and the second halfpipes of the first and second housing portions form an annular pipe (e.g., a second annular pipe). The first and second slide track portions form a sliding handle track when the first and second housing portions are brought together or moved to a closed position. The sliding handle track forms an elongated linear track having a substantially rectangular longitudinal cross-section with an open ended slot about its top surface.

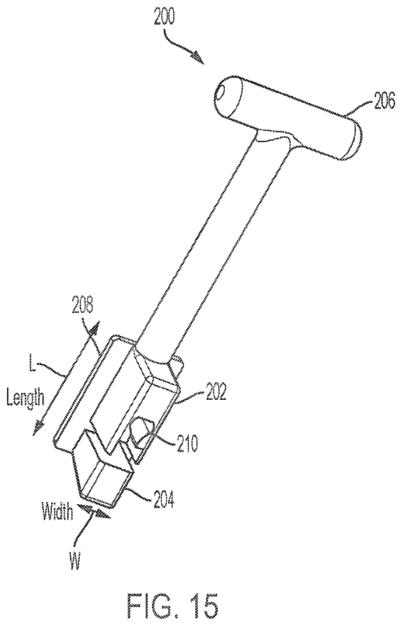

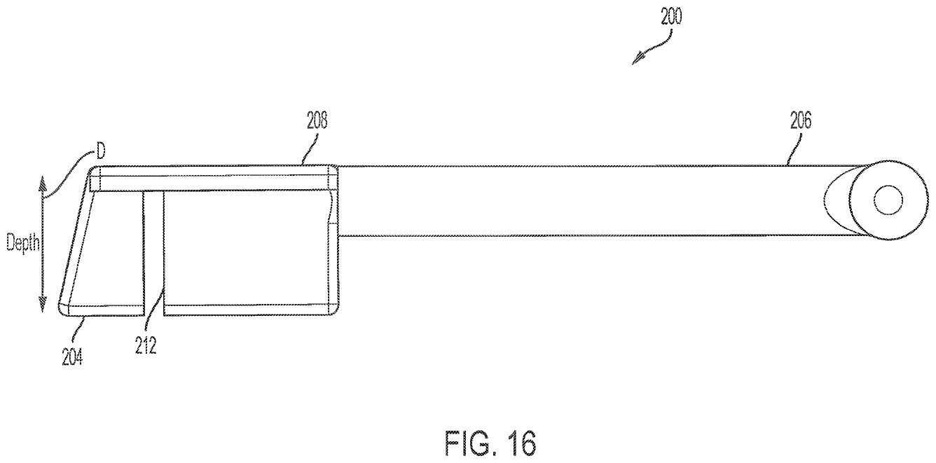

In accordance with an exemplary embodiment, the slide handle 200 is configured as best shown in FIGS. 15 and 16. The slide handle 200 is slidable within the sliding handle track (FIG. 14). The slide handle includes a main body 202, a paw 204, a handle 206, and a rail 208. The main body 202 has a substantially rectangular block shape and includes a recess 210 configured to be complementary in shape to a device its intended to receive or engage with. The device can be e.g., device 1004 of the AseptiQuik® Sterile Connector 1000 by Colder Products Company (FIG. 12). That is, the recess is sized and shaped to substantially match an overall profile of a device to be received therein. The recess 210 can extend partially a depth of the main body or extend an entirety of a depth of the main body. The recess is positioned about a distal end of the main body and in fluid communication with a distally facing end 212 of the main body.

The paw 204 is spaced from the main body 202 and spaced from the recess of the main body. The paw is configured as a linear block or hook or tab, having a depth (D) substantially the same as the depth of the main body.

The rail 208 is a substantially planar rail, but can alternatively be configured as a non-planar rail or any other shape suitable for its intended purpose. The rail is substantially rectangular in shape, from which the main body and paw extends from. The rail has a width (W) that is larger than a width of the main body and/or the paw. In particular, the rail can have a width that is 10%, 20%, 30%, 40%, 50%, 60%, 70%, 80%, 90%, 100% or 150% larger than a width of the main body and/or the paw. The rail has a length (L) that is substantially the same as an overall length that the main body and paw extends. However, the length of the rail can be shorter or longer than the overall length that the main body and paw extends, for example the rail can have a length that substantially matches a length of the paw 204.

The handle 206 extends proximally from the main body and/or the rail. That is the handle extends from a proximally facing end of the main body and/or rail, that is opposite the distally facing end of the main body. In the present exemplary embodiment, the handle is a T handle, but the handle can alternatively be another shaped handle suitable for its intended purpose, such as an U shaped handle, O shaped handle, rod shaped handle, or a shaft extending from the main body and/or the rail.

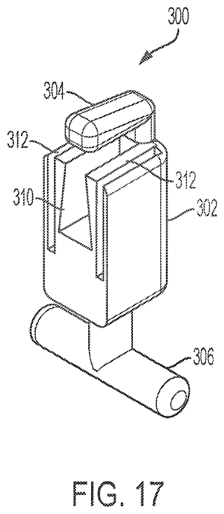

FIG. 17 illustrates another exemplary embodiment of a slide handle 300 suitable for the connection tool. The slide handle 300 includes a main body 302, a paw 304, and a handle 306. The main body 302 is substantially block shaped having a recess 310 configured to be complementary in shape to an overall profile of a device it is intended to receive or engage with. The device can be e.g., device 1004 (FIG. 12). That is, the recess is sized and shaped to substantially match an overall profile of a device to be received therein. The recess 310 can extend partially a depth of the main body or extend an entirety of a depth of the main body. The recess is positioned about a distal end of the main body and in fluid communication with a distally facing end of the main body. The main body further includes one or more slots 312 for receiving additional features of the device to be received therein.

The paw 304 is configured as a hook or hook-shaped having a depth to substantially match a depth of the main body 302, or a major percentage of the depth of the main body e.g., 50%, 60%, 70% or more.

The handle 306 is a T-shaped handle. However, the handle can alternatively be any other shaped handle suitable for its intended purpose, such as an U shaped handle, O shaped handle, rod shaped handle, or a shaft extending from the main body.

In accordance with another exemplary embodiment, the subject disclosure provides a connection tool that includes a housing having a recess for receiving a body therein, an annular tube in fluid communication with the recess, and a sliding handle track extending substantially transverse to a longitudinal central axis of the annular tube. The connection tool also includes a slide handle slidable along the sliding handle track (FIG. 1).

FIGS. 1, 18 and 19 illustrate operation of the connection tool 10 with an AseptiQuik® G (AQG) sterile connector 1000 connection device by Colder Products Company. The AQG is used to connect two ends of sterile tubing lines 1006, 1008 and utilizes a protective sheet which must be removed to fully connect the tubing lines while maintaining sterility or preventing contamination of the sterile tubing lines. One end of tubing line 1006 is inserted into the appropriate receiving nozzle of the AQG and the other end of tubing 1008 is inserted into another nozzle of the AQG. Once the tubing lines are fully seated to the AQG and the AQG is fully snapped together, the connection device (i.e., AQG) is seated within the secondary recess of the second housing portion and the pull tab 1004 is seated within the second slide track portion of the second housing portion. Alternatively the connection device can likewise be seated in the first housing portion. The tubings 1006, 1008 are seated within the first and second halfpipes of the second housing portion. Once the connection device is fully seated in the connection tool 10, the first housing portion is pivoted to the closed position (FIG. 1), which thereby allows a user to firmly and securely hold the connection device and sterile tubing lines collectively. The user secures the connection tool in the close position by manual force, or alternatively when the connection tool includes cooperating detents, the detents secures the connection tool in the closed position.

After the connection tool is moved to the closed position, the slide handle 200 is passed through the slot of the first slide track portion of the first housing portion. Specifically, the main body 202 of the slide handle is passed through the slot 28 of the first slide track portion such that the rail 208 engages a top surface of the first slide track portion or the flanges 24a, 26a of the first slide track portion. Additionally, the slide handle 200 is passed through the first slide track portion to operatively engage the pull tab 1004 of the connection device, as shown in FIG. 19, such that the various features of the pull tab are received with the recess of the slide handle and the paw engages the pull tab in a sufficient manner to allow the slide handle to pull the pull tab along the length of the sliding handle track.

In sum, the connection tool allows a user to collectively and securely hold the connection device and sterile tubing lines while pulling the pull tab in a fixed direction dictated by the sliding handle track to precisely effectuate removal of the protective sheets of the connection device thereby ensuring proper assembly of the sterile tubing lines without contamination or loss of sterility. The connection tool reduces or eliminates operator variability while making connections via connection devices. The connection tool also eliminates both horizontal and vertical movement of the connection device while the connection device pull tab is being removed.

In accordance with another exemplary embodiment, the subject disclosure provides a method of connecting tubes together. The method comprises the steps of connecting a first tube to a connection device having a pull tab, connecting a second tube to the connection device, attaching a connection tool to the connection device, the connection tool having a housing for housing the connection device and a sliding handle track. The method further comprises attaching a slide handle to the pull tab and pulling the pull tab with the slide handle along the sliding handle track.

Aspects of the present invention include:
1. A connection tool characterized by:
a housing that comprises:
   a first housing portion having:
      a first central recess for receiving a body therein, and
      a first slide track portion extending from the first central recess,
   a second housing portion movable relative to the first housing portion, the second housing portion having:
      a second central recess for receiving the body therein, and
      a second slide track portion extending from the second central recess,
   wherein the first and second slide track portions form a sliding handle track upon the first and second housing portions being moved to a closed position.
2. The connection tool of claim 1, wherein the first housing portion further comprises:
a first halfpipe in fluid communication with the first central recess; and
a second halfpipe in fluid communication with the first central recess.
3. The connection tool of claim 2, wherein the second housing portion further comprises:
a first halfpipe in fluid communication with the second central recess; and
a second halfpipe in fluid communication with the second central recess,
wherein the first halfpipes of the first and second housing portions form an annular pipe when the first and second housing portions are moved to the closed position, and the second halfpipes of the first and second housing portions form an annular pipe when the first and second housing portions are moved to the closed position.
4. The connection tool of claim 3, wherein the first slide track portion has a longitudinal axis that extends substantially perpendicular to a longitudinal axis of one of the first or second halfpipes of the first housing portion.
5. The connection tool of claim 1, wherein the first slide track portion comprises a slot extending along its entire longitudinal extent.
6. The connection tool of claim 1, wherein the first and second housing portions are pivotably connected.
7. The connection tool of claim 1, wherein the housing further comprising a hinge operatively connected to the first housing portion and the second housing portion.
8. The connection tool of claim 1, wherein the housing further comprising a living hinge operatively connected to the first housing portion and the second housing portion.
9. The connection tool of claim 1, wherein the first housing portion comprises a detent and the second housing portion comprises a cooperating detent for releasably engaging the detent of the first housing portion.
10. The connection tool of claim 1, wherein the first housing portion comprises a wing adjacent the first slide track portion.
11. The connection tool of claim 1, wherein the first housing portion comprises a left wing and a right wing, each adjacent the first slide track portion.
12. The connection tool of claim 1, further comprising a slide handle slidable within the sliding handle track.
13. The connection tool of claim 1, further comprising a slide handle that comprises:
a main body having a recess for receiving a device therein;
a paw spaced from the recess of the main body; and
a handle spaced from the main body.
14. The connection tool of claim 13, wherein the paw is a hook or tab.
15. The connection tool of claim 13, wherein the slide handle further comprising a rail having a width larger than a width of the main body.
16. The connection tool of claim 5, further comprising a slide handle that comprises:
a main body having a recess, and wherein the main body is sized to pass through the slot of the first slide track portion;
a paw spaced from the recess of the main body; and
a handle spaced from the main body.
17. A connection tool characterized by:
a housing that comprises:
   a first housing portion having:
      a first central recess for receiving a body therein,
      a first halfpipe in fluid communication with the first central recess,
      a second halfpipe in fluid communication with the first central recess,
      a first slide track portion extending substantially transverse to a central axis of the first central recess, and
      a wing adjacent the first slide track portion,
   a second housing portion pivotably connected to the first housing portion, the second housing portion having:
      a second central recess for receiving the body therein,
      a first halfpipe in fluid communication with the second central recess,
      a second halfpipe in fluid communication with the second central recess,
      a second slide track portion extending substantially transverse to a central axis of the second central recess, and
      a wing adjacent the second slide track portion,
   wherein the first and second slide track portions form a sliding handle track when the first and second housing portions are connected; and
a slide handle that comprises:
   a main body having a recess for receiving a device therein, and
wherein the main body is configured to pass along the sliding handle track,
   a paw spaced from the recess of the main body,
   a handle spaced from the main body, and
   a rail adjacent the main body.
18. A connection tool comprising:
a housing that comprises:
   a recess for receiving a body therein,
   an annular tube in fluid communication with the recess, and
   a sliding handle track extending substantially transverse to a longitudinal central axis of the annular tube; and
a slide handle slidable along the sliding handle track.
19. A method of connecting tubes together characterized by:
connecting a first tube to a connection device having a pull tab;
connecting a second tube to the connection device;
attaching a connection tool to the connection device, the connection tool having a housing for housing the connection device, and a sliding handle track;
attaching a slide handle to the pull tab; and
pulling the pull tab with the slide handle along the sliding handle track.

It will be appreciated by those skilled in the art that changes could be made to the exemplary embodiments described above without departing from the broad inventive concept thereof. It is to be understood, therefore, that this disclosure is not limited to the particular exemplary embodiments disclosed, but it is intended to cover modifications within the spirit and scope of the subject disclosure as defined by the appended claims.

1. A connection tool comprising:
a housing that comprises:
a first housing portion having:
a first central recess for receiving a body therein, and
a first slide track portion extending from the first central recess, and
a second housing portion movable relative to the first housing portion, the second housing portion having:
a second central recess for receiving the body therein, and
a second slide track portion extending from the second central recess,
wherein the first and second slide track portions form a sliding handle track upon the first and second housing portions being moved to a closed position.

2. The connection tool of claim 1, wherein the first housing portion further comprises:
a first halfpipe in fluid communication with the first central recess; and
a second halfpipe in fluid communication with the first central recess.

3. The connection tool of claim 2, wherein the second housing portion further comprises:
a first halfpipe in fluid communication with the second central recess; and
a second halfpipe in fluid communication with the second central recess,
wherein the first halfpipes of the first and second housing portions form an annular pipe when the first and second housing portions are moved to the closed position, and the second halfpipes of the first and second housing portions form an annular pipe when the first and second housing portions are moved to the closed position.

4. The connection tool of claim 3, wherein the first slide track portion has a longitudinal axis that extends substantially perpendicular to a longitudinal axis of one of the first or second halfpipes of the first housing portion.

5. The connection tool of claim 1, wherein the first slide track portion comprises a slot extending along its entire longitudinal extent.

6. The connection tool of claim 1, wherein the first and second housing portions are pivotably connected.

7. The connection tool of claim 1, wherein the housing further comprising a hinge operatively connected to the first housing portion and the second housing portion.

8. The connection tool of claim 1, wherein the housing further comprising a living hinge operatively connected to the first housing portion and the second housing portion.

9. The connection tool of claim 1, wherein the first housing portion comprises a detent and the second housing portion comprises a cooperating detent for releasably engaging the detent of the first housing portion.

10. The connection tool of claim 1, wherein the first housing portion comprises a wing adjacent the first slide track portion.

11. The connection tool of claim 1, wherein the first housing portion comprises a left wing and a right wing, each adjacent the first slide track portion.

12. The connection tool of claim 1, further comprising a slide handle slidable within the sliding handle track.

13. The connection tool of claim 1, further comprising a slide handle that comprises:
a main body having a recess for receiving a device therein;
a paw spaced from the recess of the main body; and
a handle spaced from the main body.

14. The connection tool of claim 13, wherein the paw is a hook or tab.

15. The connection tool of claim 13, wherein the slide handle further comprises a rail having a width larger than a width of the main body.

16. The connection tool of claim 5, further comprising a slide handle that comprises:
a main body having a recess, and wherein the main body is sized to pass through the slot of the first slide track portion;
a paw spaced from the recess of the main body; and
a handle spaced from the main body.

17. A method of connecting tubes together comprising:
connecting a first tube to a connection device having a pull tab;
connecting a second tube to the connection device;
attaching a connection tool to the connection device, the connection tool having a housing for housing the connection device, and a sliding handle track;
attaching a slide handle to the pull tab; and
pulling the pull tab with the slide handle along the sliding handle track.

18. A connection tool comprising:
a housing that comprises:
a recess for receiving a body therein,
an annular tube in fluid communication with the recess, and
a sliding handle track extending substantially transverse to a longitudinal central axis of the annular tube; and
a slide handle slidable along the sliding handle track.
